(19) United States
(12) Patent Application Publication
(10) Pub. No.: US 2018/0010236 A1
(43) Pub. Date: Jan. 11, 2018

(54) INHIBITORY CELL ADHESION SURFACES

(71) Applicant: METASCAPE, LLC, Maple Grove, MN (US)

(72) Inventors: DANIEL M. STOREY, LONGMONT, CO (US); LUKE J. RYVES, CREMORNE (AU); BARBARA S. KITCHELL, HOLMES BEACH, FL (US)

(21) Appl. No.: 15/689,340

(22) Filed: Aug. 29, 2017

Related U.S. Application Data
(63) Continuation of application No. 12/706,315, filed on Feb. 16, 2010, now abandoned.
Application No. 12/706,315 is a continuation-in-part of application No. 12/148,971, filed on Apr. 24, 2008, now abandoned.
(60) Provisional application No. 60/927,353, filed on May 3, 2007.

(51) Int. Cl. C23C 14/20; A61L 29/14; A61L 27/30; A61L 29/10; A61L 31/14; A61L 31/08; C23C 14/32; A61L 27/50; A61F 2/30

(52) U.S. Cl. CPC C23C 14/20; C23C 14/325; A61L 29/14; A61L 27/306; A61L 27/50; A61L 29/106; A61L 31/14; A61L 31/088; A61F 2/3094; A61F 2/30767; A61L 2400/12; A61L 2400/18

(57) ABSTRACT

Nanostructured surfaces on selected substrates are described which are highly resistant to cell adhesion. Such surfaces on medical implants inhibit fibroblast adhesion particularly on nanorough titanium deposited on smooth silicone surfaces. The nanostructured deposited metal coatings can also be engineered so that several cell types, including endothelial, osteoblast, and fibroblast cells, show little if any tendency to attach to the coated surface in vivo.

This application is a continuation of U.S. application Ser. No. 12/706,315, filed Feb. 16, 2010, which is a continuation-in-part of U.S. application Ser. No. 12/148,971, filed Apr. 24, 2008, which claims the benefit of U.S. provisional application Ser. No. 60/927,353, filed May 3, 2007.

### BACKGROUND OF THE INVENTION

### Field of the Invention

The invention relates generally to the field of engineered surfaces, particularly to surfaces modified for increased resistance to cell adhesion.

### Description of Background Art

Inhibition of cell adhesion on various surfaces is a particularly important goal in the design of certain medical devices, particularly those devices where obstruction or cell proliferation is undesirable. Such devices used in vivo are susceptible to undesirable cell adherence and proliferation. Implants, vascular prostheses and kidney dialysis equipment are especially prone to undesirable overgrowths of soft tissue cells such as fibroblasts, resulting in failure of the device and need for short term replacement.

Studies on surface modifications are typically designed to identify materials that enhance cell adhesion; for example, enhancement of osteointegration on implanted titanium alloys or RGD-coated titanium implants (Elmengaard, et al., 2005). Bioactive proteins such as collagen and fibronectin have been attached to dental implants in order to enhance gingival fibroblast binding and enhance sealing of soft tissue to implant surfaces.

Yet cell adhesion and proliferation remains a concern for many types of implants and devices used in contact with tissue or body fluids. For example, coatings have been employed on intraocular lenses in order to lessen damage to endothelial cells when the lenses are inserted as well as to mitigate formation of biofilms. Different plastic coatings deposited from a plasma reactor onto the lens have been used to provide defined thickness coatings of selected polymers on poly(methylacrylate). Film materials included perfluropropane, ethylene oxide, 2-hydroxyethyl methacrylate and N-vinyl-2-pyrrolidone (Mateo and Ratner, 1989).

In efforts to prevent cells from adhering to glass surfaces, Owens, et al. (1987) studied a large number of polymers coated on glass for ability to prevent adhesion by red blood cells, Dictyostelium discoideum amoebae and Escherichia coli. Polyethylene oxide (PEO) for example was already known to have anti-adhesive properties used either alone or as a co-polymer, as demonstrated by lack of adhesion of platelets and rabies virus on coated glass. The researchers tested several co-polymer coatings on glass using polymers that had hydrophobic and hydrophilic segments. The three types of cells tested in vitro were readily washed from a hydrophobic glass surface coated with a bifunctional F-106 Pluronic, demonstrating lack of adhesion even after 1 hr exposure to the cells.

More recently Ishihara, et al. (1999) studied fibroblast adhesion and proliferation on polymer coated poly(ethylene terephthalate) substrates. Cell adherence appeared to be related to the hydrophobicity of the coated surface, in turn determined by the composition of the co-polymer. Polymers poly 2-methacryloyloxyethyl phosphorylcholine (MPC)-co-n-butyl methacrylate copolymer and poly(2-hydroxyethylmethacrylate) were tested. Higher amounts of MPC in the one copolymer led to a weakening of the interaction between the polymer surface and adhering proteins and consequently a decrease in the number of fibroblast cells adhering to those surfaces.

Medical devices and implants that remain in the body for any period of time tend to act as foci of inflammation, due in part to adherence and build up of fibrous tissue when fibroblasts proliferate on the surfaces of orthopedic or vascular implants. There is a need for methods of modifying surface characteristics of materials used in vivo so that undesirable cell attachment does not occur.

Ogawa (2006) describes microstructuring a substrate surface by a “controlled nanostructuring process that allows the creation of nanostructure on the top of the existing microstructure of the substrate.” Nanostructured titanium for example is deposited onto a microstructured metal or nonmetal surface. Titanium implants with nanosphere surface structure are mechanically superior to unstructured surfaces in push-pull tests.

### SUMMARY OF THE INVENTION

The present invention concerns a process for treating surfaces to significantly reduce cell adhesion compared with the unmodified surface. The nonadherent surfaces are illustrated with several different substrate materials and with different types of cells, including fibroblasts, endothelial and osteoblast cells.

An important feature of the invention is the preparation of structured surfaces that effectively change surface energy and hydrophobicity. Surface energy can be increased so that cell adherence is significantly weakened. As disclosed herein, the described structured surfaces do not promote cell adhesion or, if cells are adhered, will readily disengage from the surface; for example, in situations where laminar flow is involved such as on surfaces of medical implants exposed to blood flow in vivo.

In particular, it is shown that activated surfaces can be created on the surface of a selected substrate, metal or non-metal, thereby raising surface energy and significantly decreasing cell adhesion and proliferation. An example is the controlled titanium plasma treatment of a silicone surface. When fibroblast adhesion to the treated surface was tested, cell density was decreased over 50% compared with adhesion to untreated silicone surfaces. In contrast, titanium deposition on polytetrafluoroethylene (PTFE) and ultra high molecular weight polyethylene (UHMWPE) substrates under different titanium plasma exposure conditions lowered rather than increased surface energy, resulting in up to a 180% increase in cell density on the treated surface compared with the untreated surface.

Surface energy can be increased or decreased for virtually any surface using a controlled plasma surface treatment procedure. Generally, this requires creating a plasma and controlling macromolecule deposition on a selected surface. The size and distribution of the macromolecules determines surface energy and hydrophobicity of the surface. In effect, ion plasma deposition (IPD) can be used to increase surface area on selected substrates. Under selected conditions, this results in higher surface energy, increased hydrophobicity and decreased cell adherence compared to untreated surfaces.

Surfaces of bone and vascular implants are particularly susceptible to in vivo cell adhesion. Materials currently used for medical implants include titanium, titanium alloys such as Ti6Al4V and CoCrMo alloys, silicone, polyethylene and the like. The surface treatments disclosed herein can be adapted to texturize a substrate surface so that cell adhesion is significantly reduced depending on nanoroughness of the surface.

Changes in surface characteristics as a result of using the disclosed surface treatment were assessed by measuring the dynamic contact angle. Increased or decreased contact angles on treated surfaces were exhibited by water droplets depending on the treatment conditions. Treatment of a silicone surface with plasma generated titanium nanoparticles caused increased water contact angles with the surface. On the other hand, selectively modifying the titanium generated plasma exposure on UHMWPE and PTFE resulted in decreased water contact angles with the treated surface compared with untreated surfaces. When water droplet contact angles were decreased, there was increased cell adhesion and proliferation on the surfaces.

The invention shows that nanostructured surfaces on a substrate play a role in cell adhesion and that under selected conditions, illustrated with ion plasma deposited titanium on smooth silicone, cell adherence can be modified such that increased or decreased adhesion is obtained compared to adhesion on the unmodified substrate surface. While decreased cell adhesion was seen on nanostructured titanium surfaces on smooth silicone using controlled IPD parameters, and not on UHMWPE and PTFE titanium coated surfaces, the underlying substrate surface may also be a factor in combination with selected deposition conditions, as described herein. Metals other than titanium, including titanium alloys, deposited under selected conditions, are expected to provide decreased cell adhesion on smooth silicone and other substrates with surfaces similar to smooth silicone.

### BRIEF DESCRIPTION OF THE FIGURES

FIG. 1 shows decreased fibroblast adhesion on a titanium treated silicone (Si) surface compared with UHMWPE and PTFE titanium treated and untreated (Si—C, UHMWPE-C and PTFE-C) surfaces. ⁺=p<0.01 compared with corresponding untreated sample.

FIG. 2A shows fibroblast proliferation measured as increased cell density on titanium treated silicone, UHMWPE and PTFE after one day in vitro exposure to fibroblast cells compared with untreated surfaces.

FIG. 2B shows fibroblast proliferation measured as increased cell density on titanium treated silicone, UHMWPE and PTFE after three days in vitro exposure to fibroblast cells compared with untreated surfaces.

FIG. 2C shows fibroblast proliferation measured as increased cell density on titanium treated silicone, UHMWPE and PTFE after five days in vitro exposure to fibroblast cells compared with untreated surfaces.

FIG. 3 shows fluorescent images comparing fibroblast proliferation on a titanium plasma treated and untreated silicone, PE and PTFE surfaces. DAPI dye under fluorescent microscope was used for cell counting. Cell nuclei were observed as fluorescing dots. Data indicated that uncoated silicone has a higher cell density compared to titanium coated silicone. Day 3 (or day 5).

FIG. 4 shows the general features of a modified cathodic arc IPD apparatus: target 1; substrate 2; movable substrate holder 3; vacuum chamber 4; power supply 5 for the target; and arc control 6 to adjust speed of the arc.

FIG. 5 shows the water droplet contact angles for silicone (Si), polyethylene (PE), and Teflon® (PTFE) for untreated surfaces and for titanium plasma treated surfaces (Si—C, PE-C and PTFE-C).

FIG. 6 is a graph showing GF and PLF cell adhesion on titanium coated smooth silicone. Each test was on a titanium coated substrate run under the parameters listed in Table 1. Percent change in cell adhesion is determined by comparison with the number of cells adhered to an uncoated smooth silicone substrate surface after 7 days incubation.

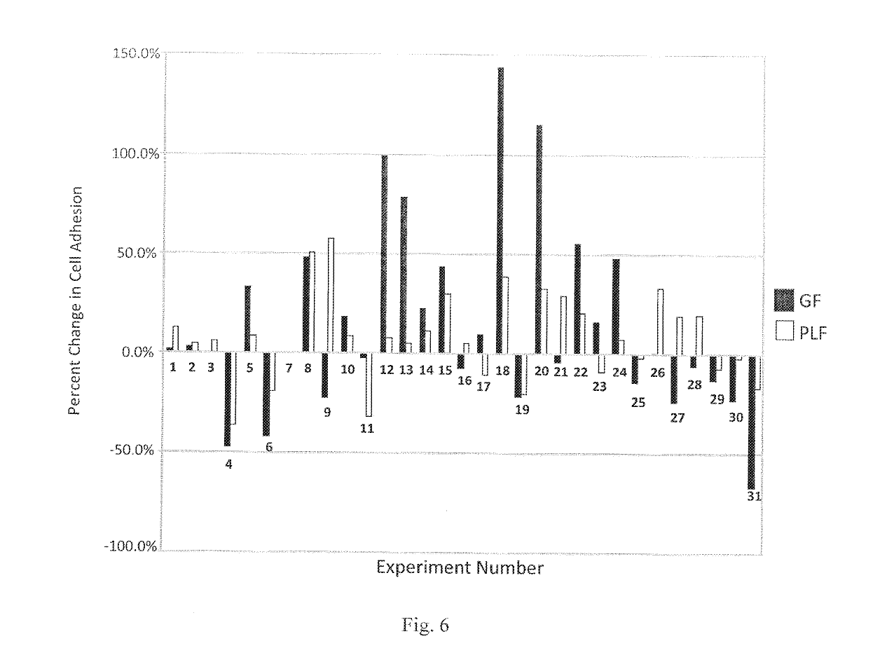

### DETAILED DESCRIPTION OF THE INVENTION

The present invention provides controlled nano-textured surfaces particularly suitable for medical implant surfaces where in vivo cell adhesion is undesirable. A surface treatment has been developed that has the ability to decrease the attachment of osteoblast, endothelial, and fibroblast cells to the treated surfaces compared to cell attachment on the untreated surface. In particular, ion plasma deposited titanium on a smooth silicone surface shows a significant decrease in cell adhesion compared to IPD titanium on other surfaces such as polyethylene or polytetrafluoroethylene.

By exposing a surface to an activated metal plasma that can be controlled to change hydrophobic characteristics of the surface, the surface energy of a substrate can be raised. This results in the inhibition of attachment of various types of cells, including endothelial, osteoblast and particularly fibroblast cells. This contrasts to literature reported observations indicating that low surface energy generally contributes to increased cell attachment (Curtis, et al., 2004; Webster, et al., 1999).

In order to provide surfaces that inhibit cell adhesion, a selected metal or polymer substrate surface is exposed to an IPD produced metal plasma under defined conditions. Surface hydrophobicity is increased in a manner that appears to be related to the size of ion particulates on the substrate surface and the resulting surface texturing caused by the deposited metal. Deposition conditions can be adjusted to control the size of nanoparticles that contact and texture the surface. The nanoparticle deposition treatment, performed under certain defined conditions, is able to increase the surface area and raise surface energy, thereby increasing hydrophobicity and significantly decreasing cell adherence.

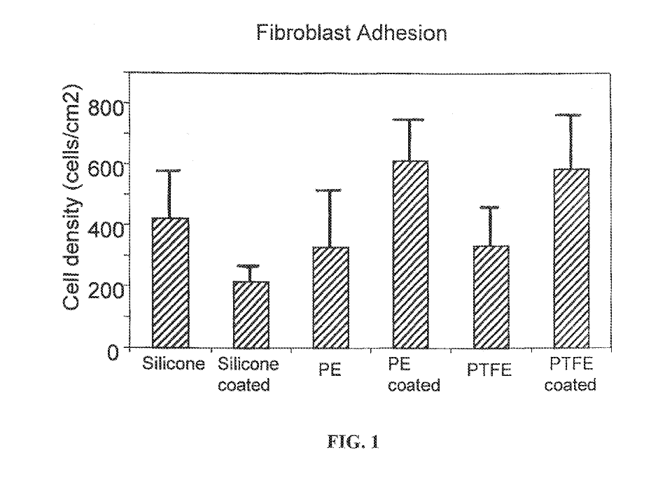

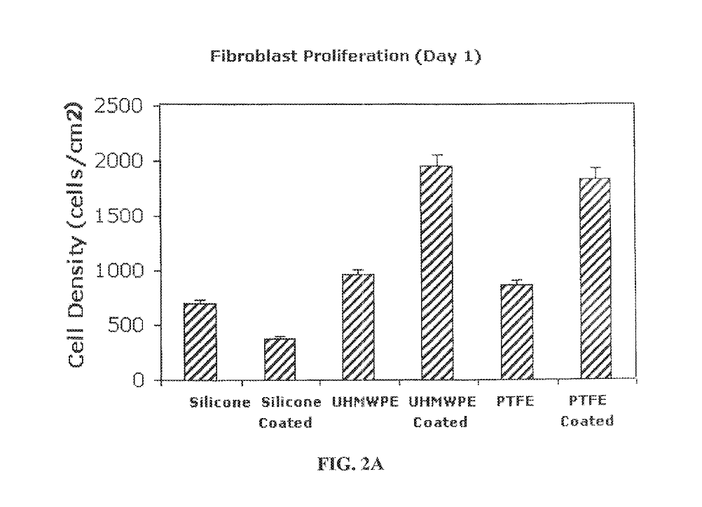

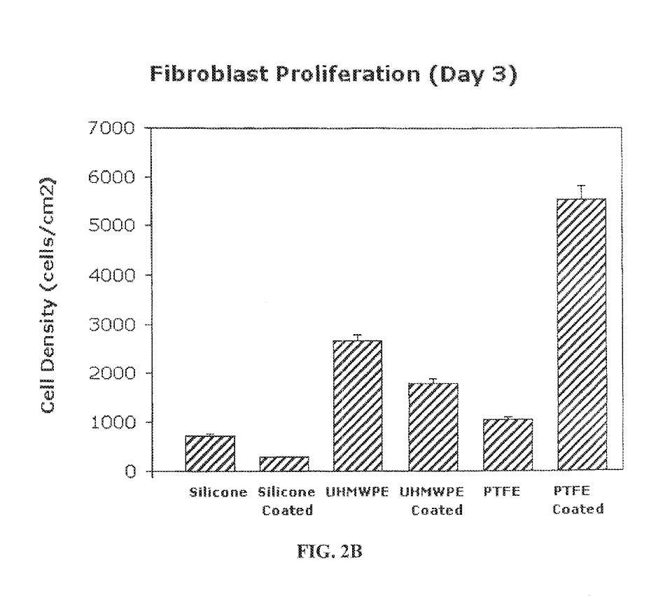

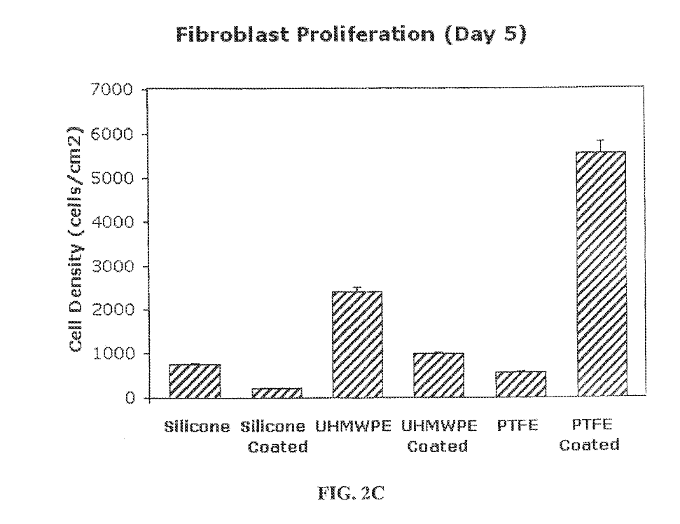

Effects on surface energy have been demonstrated for various materials in relation to the surface treatment. As seen in FIG. 1, a smooth silicone substrate surface was treated so that the surface energy of the nanorough titanium surface was increased, while the same modification of the plasma treatment conditions for UHMWPE and PTFE resulted in a decrease in surface energy compared with the untreated surfaces. Significantly decreased fibroblast density after 1, 3 and 5 days on the treated silicone surface was observed in contrast with the highly increased cell densities observed on treated UHMWPE and PTFE surfaces as shown in FIGS. 2A, 2B and 2C.

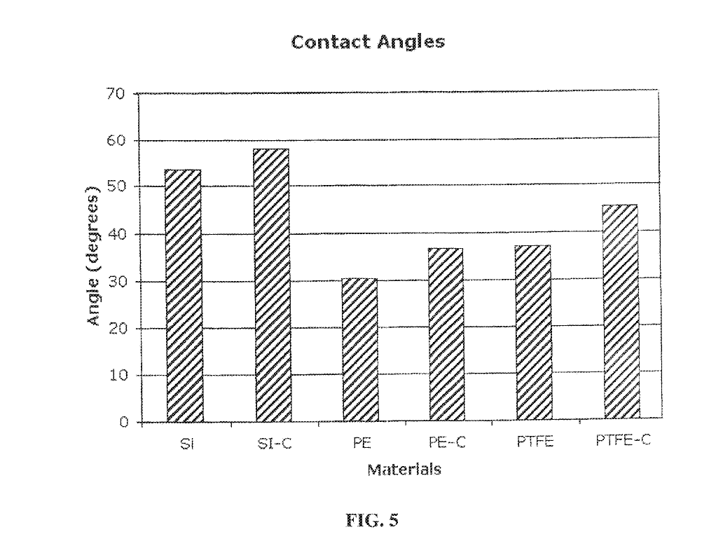

Measurement of the contact angle on the titanium treated silicone, UHMWPE and PTFE surfaces using a water droplet showed that the contact angle increased on the titanium coated silicone surface but decreased on the other treated surfaces compared with the respective untreated surfaces. An increase in contact angle indicates an increase in surface energy, and thus the hydrophobicity. The decrease in hydrophobicity on the UHMWPE and PTFE coated surfaces correlates with the increased cell adhesion on those surfaces compared with the decreased adhesion observed on the titanium treated silicone surface. FIG. 5 shows the contact angle measurements for a water droplet on treated and untreated silicone, UHMWPE and PTFE surfaces.

The texturing and treatment of the different substrate surfaces utilized a titanium ion plasma deposition (IPD) process. This process creates nano-rough nanoparticles on the surface of the substrates, thus changing the surface energy and creating a more hydrophobic surface. Basic procedures for creating a nano-rough surface can be found in Webster, et al. (2006).

Controlling the texturing of a wide range of materials using a customized IPD process provides control of the surface energy of any material. Because the surface treatment is independent of the substrate, this ability to control surface hydrophobicity and therefore cell adhesion characteristics will be applicable to any material.

The size of the nano texturing (i.e., particle size) directly controls the surface energy and the hydrophobicity. Thus, the IPD process can be adjusted to control the physical characteristics of the nano texturing such that in effect the surface energy of virtually any substrate can be engineered.

Materials

Silicone sheets, bars and strips were purchased from McMaster-Carr (Aurora, Ohio 44202-8087) as Material Type NSP certified silicone rubber bar sheets, bars or strips without backing, with a thickness of ¼″, temperature range of −75° F. to +400° F., smooth finish meeting FDA and NSF, FDA compliant and NSF 51 certified, sold under part number 5827T34.

Fibroblasts (purchased from ATCC) were grown in culture until confluence in DMEM with 10% FBS and 1% P/S. Material samples were used as supplied. Before cell experiments, samples were sonicated and autoclaved.

Endothelial cells (purchased from ATCC) were grown in culture until confluence in DMEM with 10% FBS and 1% P/S. Material samples were used as supplied. Before cell experiments, samples were sonicated and autoclaved.

Osteoblasts (purchased from ATCC) were grown in culture until confluence in DMEM with 10% FBS and 1% P/S.

### EXAMPLES

The following examples are provided as illustrations of the invention and are in no way to be considered limiting.

### Example 1—Ion Plasma Deposition

Ion Plasma Deposition (IPD) is a method of creating highly energized plasma using a cathodic arc discharge created from a target material, typically solid metal. An arc is struck on the metal and the high power density on the arc vaporizes and ionizes the metal, creating a plasma which sustains the arc. A vacuum arc is different from a high pressure arc because the metal vapor itself is ionized, rather than an ambient gas.

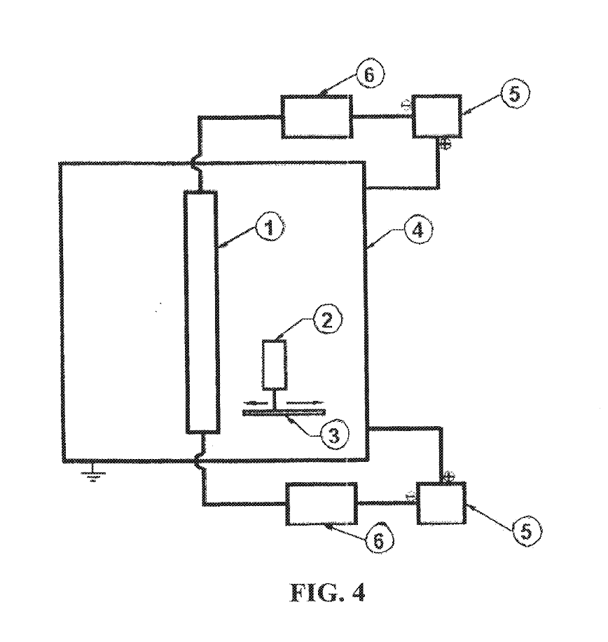

FIG. 4 illustrates an apparatus suitable for controlling deposition of the plasma ejected from the cathodic arc target source 1 onto a substrate 2. The size of the particle deposited, and thus the degree of nanotexturing of the deposited surface is controlled by a movable substrate holder 3 within the vacuum chamber 4 or by a power supply 5 to the target and adjustment of arc speed 6. The closer a substrate is to the arc source, the larger and more densely packed will be the particles deposited on the substrate.

Control of the substrate position with respect to the target and arc speed allow precise control of the surface characteristics of the substrate with respect to density, number and size of the nanoparticles arranged in the substrate surface. This in turn determines the surface area of the substrate and affects hydrophobic properties of the substrate surface. Hydrophobicity of a nanoparticle textured surface can be determined by measurement of the contact angle of a water droplet on the surface.

### Example 2—Fibroblast Attachment/Repulsion

Three types of substrates were treated with Ti 6-4 using the described IPD process to form a deposit with random depth up to 200 nm. The average nano-particle size of the coating was 10 to 30 nanometers and was confirmed by SEM analysis.

Fibroblasts were seeded onto each substrate at 3500 cells/cm². Samples were first placed in 12 and 24 well cell culture plates. 175 μl of cell-containing droplets in media were added to the samples before incubating at 37° C. and 5% CO₂ for 4 hours. The samples were washed 3 times with PBS, fixed in formaldehyde for 10 min, and again washed in PBS 3 times. Cells were then counted using fluorescent microscopy and DAPI dye. Images of cell morphology were also acquired. Experiments were conducted in triplicate with two repeats each (total of six samples for each averaged data point). Standard statistical analysis by Student t-test was used to determine differences between substrates.

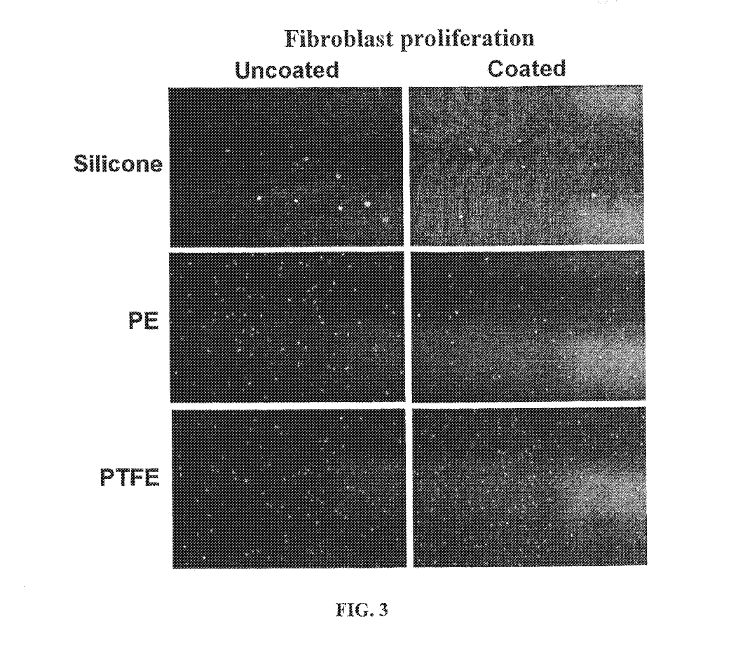

Results showed an unexpected decrease in in vitro fibroblast adhesion on nanorough titanium surfaces on smooth silicone compared to all other samples tested (FIG. 3) at one, three and five days after exposure to fibroblast cells. This indicated that less adhesion of fibroblasts on implant surfaces will result in less soft and scar tissue formation around either an orthopedic or vascular implant that is coated with nanostructured titanium on a silicone surface.

Qualitative fibroblast morphology images matched the quantitative data showing less fibroblast adhesion on titanium coated silicone. Specifically, less well-spread cells were observed on titanium coated silicone compared to other substrates tested.

### Example 3: Decreased Endothelial Cell Adhesion on Titanium Coated Silicone

Silicone was treated with Ti 6-4 using the IPD process to form textured thicknesses up to 200 nm. The average nano-particle size of the coating was 30 to 40 nanometers and was confirmed by SEM analysis.

Endothelial cells were seeded onto each substrate at 3500 cells/cm². Samples were first placed in 12 and 24 well cell culture plates. 175 μl of cell-containing droplets in media were added to the samples and were incubated at 37° C. and 5% CO₂ for 4 hours. The specimens were then washed 3 times with PBS, fixed in formaldehyde for 10 min, and again washed three times in PBS. Cells were then counted using fluorescent microscopy and DAPI dye. Images of cell morphology were also acquired. Experiments were conducted in triplicate with two repeats each (total of six samples for each averaged data point). Standard statistical analysis by Student t-test was used to determine differences between substrates.

Results showed a decrease in cell adhesion on the coated silicone parts of approximately 25%.

### Example 4—Decreased Osteoblast Proliferation on Titanium-Coated Surfaces

Three types of substrates were treated with Ti 6-4 using the IPD process. The average nano-particle size on the surface was 10 to 30 nanometers and was confirmed by SEM analysis.

Purchased substrate samples were used as supplied. The samples were trimmed with a razor to make the adhesion surface flat. Before cell experiments, samples were sonicated in 70% ethanol and autoclaved or UV treated for 20 minutes.

Osteoblasts were seeded onto each substrate at 3500 cells/cm², then placed in 12 and 24 well cell culture plates. 175 μl of cell-containing droplets in media was added to the samples and then incubated at 37° C. and 5% CO₂ for 4 hours. At the end of the 4 hours the cell containing droplets were removed and each well with a sample filled with DMEM media and incubated again under the same conditions for 1, 3, and 5 day proliferation. Specimens were then washed 3 times with PBS, fixed in formaldehyde for 10 min, and again washed in PBS 3 times after 24, 72, and 120 hours respectively. Cells were counted using fluorescent microscopy and DAPI dye. Images of cell morphology were also be acquired. Experiments were conducted in triplicate with two repeats each (total of six samples for each averaged data point). Standard statistical analysis by Student t-test were used to determine differences between substrates.

Results of the 1, 3 and 5 day test are expected to show decreased osteoblast proliferation on all coated substrates over their uncoated counterparts as was shown for fibroblast cells (see FIG. 3).

### Example 5—Decreased Cell Attachment Using IPD Surface Treatment

Ion plasma deposition was used to modify a silicone surface by depositing titanium to create a nanoparticulate textured nano-rough surface. The roughness characteristics of nanostructured titanium surfaces that enhance cell adherence have been reported (Webster, et al., 2004) but these surfaces, while produced from an ion plasma, are different in structure and physical characteristics from the nanorough surfaces tested in this example which show decreased cell adherence.

Several nano-structured titanium surfaces were prepared and tested for hydrophobicity and surface energy. Different types of cells were expected and did in fact show varying degrees of adhesion.

### Example 6—Controlled Increase of Surface Energy

This example showed that controlled deposition of nanoparticles on selected surfaces will affect and can be used to change surface energy. As illustrated in FIG. 1 and FIGS. 2A-C, silicone treated under IPD conditions to increase surface energy showed little tendency for fibroblast adherence, while UHMWPE and PTFE, each treated to lower surface energy, exhibited increased fibroblast adherence compared with the respective untreated surfaces.

In a 4 hr fibroblast adhesion assay, droplets containing 3500 cells/cm² were incubated on silicone, UHMWPE and PTFE titanium coated surfaces. After incubation, the samples were washed with PBS and the cells fixed with formaldehyde and stained with DAPI dye. Titanium treated UHMWPE and PTFE surfaces exposed in vitro to fibroblasts resulted in higher fibroblast densities on the treated surfaces compared to uncoated surfaces, while titanium treated silicone surfaces had a lower density of cell adhesion compared with the uncoated material. Data show a mean plus/minus standard deviation where *p<0.01 compared with the uncoated counterpart.

FIG. 5 compares surface contact angle of a water droplet on silicone, UHMWPE and PTFE surfaces treated with IPD titanium, showing that the surface treatment used on the silicone surface had a higher surface energy resulting in lower cell adherence as indicated by the increased contact angle on the titanium coated silicone surface compared to the decreased contact angle for UHMWPE and PTFE relative to their uncoated surfaces.

### Example 7—Periodontal Ligament Fibroblast and Gingival Fibroblast Adhesion on Nanostructured Titanium Coated Smooth Silicone Substrates

Smooth silicone substrates were purchased from McMaster-Carr (Aurora, Ohio). Tests 1-31 (Table 1) were conducted on titanium coated smooth silicone surfaces after 7 days incubation with GF and/or PLC cells. Test 32 is a control measuring change in cell adhesion on untreated smooth silicone surfaces after incubation for 7 days.

Table 2 showing % change is based on determining the number of cells attached to the surface of a smooth silicone substrate control and dividing the number on the titanium treated substrate by the “control” number. For example, 1000 cells on the smooth silicone surface of the control and 500 on the titanium coated silicone substrate represents a 50% reduction in cell adhesion.

The titanium coated silicone substrates were prepared by ion plasma deposition of titanium using an apparatus substantially as illustrated in FIG. 4. All coated substrates were exposed to the titanium deposition for 10 min. In reference to Table 1, the switch speed is the speed at which the arc is switched from one end of the target to the other in cycles per second. Distance is distance from the cathode (e.g., if the silicone substrate is 12″ from the cathode surface, the distance is 12″). Pressure in the vacuum chamber is the pressure of the chamber during the deposition/modification of the substrate. This can be kept constant by addition of an inert gas such as argon (Ar). Bias is the voltage put on the parts during the deposition, always in negative voltage Current is the current of the arc or the current supplied to the target.

TABLE 1

| Run | Current | Bias | Pressure | Distance | Switching Speed |
|---|---|---|---|---|---|
| # | (A) | (V) | (mT) | (in) | (Hz) |
| 1 | 100 | 0 | 1 | 6 | 0.25 |
| 2 | 300 | 0 | 1 | 6 | 0.25 |
| 3 | 100 | 200 | 1 | 6 | 0.25 |
| 4 | 300 | 200 | 1 | 6 | 0.25 |
| 5 | 100 | 0 | 5 | 6 | 0.25 |
| 6 | 300 | 0 | 5 | 6 | 0.25 |
| 7 | 100 | 200 | 5 | 6 | 0.25 |
| 8 | 300 | 200 | 5 | 6 | 0.25 |
| 9 | 100 | 0 | 1 | 24 | 0.25 |
| 10 | 300 | 0 | 1 | 24 | 0.25 |
| 11 | 100 | 200 | 1 | 24 | 0.25 |
| 12 | 300 | 200 | 1 | 24 | 0.25 |
| 13 | 100 | 0 | 5 | 24 | 0.25 |
| 14 | 300 | 0 | 5 | 24 | 0.25 |
| 15 | 100 | 200 | 5 | 24 | 0.25 |
| 16 | 300 | 200 | 5 | 24 | 0.25 |
| 17 | 100 | 0 | 1 | 6 | 5 |
| 18 | 300 | 0 | 1 | 6 | 5 |
| 19 | 100 | 200 | 1 | 6 | 5 |
| 20 | 300 | 200 | 1 | 6 | 5 |
| 21 | 100 | 0 | 5 | 6 | 5 |
| 22 | 300 | 0 | 5 | 6 | 5 |
| 23 | 100 | 200 | 5 | 6 | 5 |
| 24 | 300 | 200 | 5 | 6 | 5 |
| 25 | 100 | 0 | 1 | 24 | 5 |
| 26 | 300 | 0 | 1 | 24 | 5 |
| 27 | 100 | 200 | 1 | 24 | 5 |
| 28 | 300 | 200 | 1 | 24 | 5 |
| 29 | 100 | 0 | 5 | 24 | 5 |
| 30 | 300 | 0 | 5 | 24 | 5 |
| 31 | 100 | 200 | 5 | 24 | 5 |
| 32 | 0 | 0 | 0 | 0 | 0 |

7 day test—Prior to use, all substrates were sterilized under UV light overnight. PLF (ScienCell Research Laboratories, Carlsbad, Calif.; population numbers 5-7) and GF (CRL-2014 American Type Culture Collection, population numbers 5-7) in Dulbecco's Modified Eagle Medium (Gibco) supplemented with 10% fetal bovine serum (Hyclone) and 1% Penicillin/Streptomycin (Hyclone) were seeded (at a density of 3500 cells/cm² for HPLF and 1500 cells/cm2 for HGF) onto the substrate of interest (silicone) and were then placed in standard cell culture conditions (humidified, 5% CO₂/95% air environment). After the prescribed time period (7 days), substrates were rinsed in phosphate buffered saline to remove any nonadherent cells. The remaining cells were fixed with formaldehyde (Aldrich), stained with Hoescht 33258 dye (Sigma), and counted under a fluorescence microscope (Leica, DM IRB). Five random fields were counted per substrate. All substrates were run in triplicate Standard t-tests were used to check statistical significance between means. Cells were then counted using fluorescent microscopy and DAPI dye. Images of cell morphology were also acquired. Experiments were conducted in triplicate with two repeats each (total of six samples for each averaged data point). Standard statistical analysis by Student t-test was used to determine differences between substrates.

Peridontal Ligament Fibroblast (PLF) and Gingival fibroblast (GF) cell lines were used to test adhesion on the titanium nanostructured surfaces prepared by IPD deposited titanium on the smooth silicone substrates. 31 adhesion tests were performed using different process parameters to deposit the titanium as shown in Table 1. Percent changes in cell adhesion of PLF and GF cells compared to the number of cells adhered to smooth uncoated silicone substrates were measured after seven days. Both types of fibroblast cells showed increased adherence in tests 1-3, 5, 8, 10, 12-15, 18, 20, 22, and 24, Both PLF and GF cells showed decreased adherence after 7 days in tests 4, 6, 11, 19, 25, and 29-31. No change for either PLF or GF cells was observed after 7 days in test 7. Table 2 shows approximate percent changes in cell adhesion compared to the control after 7 days incubation.

TABLE 2

| | % Increase/decrease | % Increase/decrease |
|---|---|---|
| Test | GF adhesion* | PLF adhesion* |
| 1 | <5 | 12 |
| 2 | <5 | 5 |
| 3 | 0 | 7 |
| 4 | −30 | −30 |
| 5 | 35 | 8 |
| 6 | −38 | −30 |
| 7 | 0 | 0 |
| 8 | 48 | 50 |
| 9 | −20 | 60 |
| 10 | 18 | 5 |
| 11 | −5 | −25 |
| 12 | 100 | 5 |
| 13 | 78 | 5 |
| 14 | 25 | 10 |
| 15 | 40 | 30 |
| 16 | −5 | 5 |
| 17 | 10 | −10 |
| 18 | 145 | 35 |
| 19 | −20 | −20 |
| 20 | 115 | 30 |
| 21 | −5 | 30 |
| 22 | 55 | 20 |
| 23 | 15 | −10 |
| 24 | 50 | 5 |
| 25 | −15 | −5 |
| 26 | 0 | 30 |
| 27 | −20 | 20 |
| 28 | −5 | 20 |
| 29 | −10 | −5 |
| 30 | −2− | 0 |
| 31 | −70 | −20 |
| 32 | Control 0 | Control 0 |

*% increase or decrease is change in number of cells adhered to the titanium coated silicon substrate compared to uncoated smooth silicon (control 32) after 7 days incubation with the cells.

### REFERENCES

Elmengaard, B., Bechtold, J. E. and Soballe, K., J., “In vivo effects of RGD-coated titanium implants inserted in two bone-gap models”, Biomedical Materials Research, Part A, v. 75A, (2), 249-255 (2005).
Mateo, N. B. and Ratner, B. D., “Relating the surface properties of intraocular lens materials to endothelial cell adhesion damage”, Investigative Ophthalmology & Visual Science, v. 30 (5), May 1989, 853-860.
Owens, N. F., Gingell, D. and Rutter, P. R., “Inhibition of cell adhesion by a synthetic polymer adsorbed to glass shown under defined hydrodynamic stress”, P. R., J. Cell Sci. 87, 667-675 (1987).
Ishihara, K., Ishikawa, E., Iwasaki, Y. and Nakabayashi, N., “Inhibition of fibroblast cell adhesion on substrate by coating with 2-methacryloyloxyethyl phosphorylcholine polymers”, Biomater. Sci. Polym. Ed., 10(10), 1047-61 (1999).
Webster, et al. BSME Conference, Chicago, Ill., October 2006.
Ogawa, International Publication No. WO 2006/102347 A2 (28 Sep. 2006).
Curtis, A. S. G., Gadegaard, N., Dalby, M. J. Riehle, M. O., Wilkinson, C. D. W. and Aitchison, G., “Cells react to nanoscale order and symmetry in their surroundings”, IEEE Trans Nanobiosci, 3, 61-65 (2004).
Webster, T. J., Siegel, R. W., and Bizios, R., “Osteoblast adhesion on nanophase ceramics”, Biomaterials 20, 1221-1227 (1999).

What is claimed is:

1. An implant device comprising:
a substrate having a surface; and
a nanostructured coating applied to the surface, the coating resulting in a contact angle greater than about 50 degrees, wherein the value of the contact angle is such that cell attachment to the coating is reduced compared to cell attachment to an uncoated surface.

2. The implant according to claim 1, wherein the contact angle is higher for the coated surface than for the uncoated surface.

3. The implant according to claim 1, wherein the uncoated surface results in a contact angle less than 50 degrees.

4. The implant device according to claim 1, wherein the substrate comprises a non-metal.

5. The implant according to claim 1, wherein the coating results in a surface energy value, wherein the value of the surface energy is such that cell attachment to the coating is reduced compared to cell attachment to an uncoated surface.

6. The implant according to claim 5, wherein the surface energy value is higher for the coated surface than for the uncoated surface.

7. The implant according to claim 1, wherein the reduction of cell attachment is exhibited by fibroblast, endothelial, and osteoblast cells.

8. The implant according to claim 7, wherein the fibroblast cell comprises a periodontal ligament fibroblast (PLF), gingival fibroblast (GF), or a mixture of PLF and GF cells.

9. The implant according to claim 1, wherein the cell attachment to the coated surface is an order of magnitude less than cell attachment to the uncoated surface.
